(19) United States
(12) Patent Application Publication
(10) Pub. No.: US 2003/0032620 A1
(43) Pub. Date: Feb. 13, 2003

(54) Low molecular weight chondroitin sulphate compound having cosmetic activity

(76) Inventors: Annie Landrein, Nantes (FR); Philippe Roy, Nantes (FR); Patrick Durand, Reze (FR); Charles Delannoy, Wimereux (FR)

Correspondence Address: JACOBSON HOLMAN, PROFESSIONAL LIMITED LIABILTY COMPANY, 400 SEVENTH STREET, N.W., WASHINGTON, DC, 20004, US

(21) Appl. No.: 09/912,267

(22) Filed: Jul. 25, 2001

(51) Int. Cl. A61K 31/737; C08B 37/00

(52) U.S. Cl. 514/54; 536/54

(57) ABSTRACT

The invention relates to a low molecular weight chondroitin sulphate compound having cosmetic activity, characterised more particularly by efficient incorporation in vitro of thymidine, glucosamine and leucine in fibroblast macromolecules of the human cutis. Local application of this compound stimulates fibroblast metabolism. The invention also relates to a method of preparing the said compound.

### SUBJECT OF THE INVENTION

The invention relates to a low molecular weight chondroitin sulphate compound having cosmetic activity. It belongs to class A61K and sub-classes 7/36-7/40-7/48-33/06-33/32 and 35/56 of the international classification (6^{th} edition)

### PREAMBLE

The constituents of the extracellular matrix of the skin are divided among various categories mainly comprising laminin, the various kinds of collagens, elastin, fibronectin and various proteoglycanes, the main ones being heparan sulphate and chondroitin sulphates. These proteoglycanes (J. T. Gallagher, 1989. Current Biol., 1, 1201) comprise a protein part on to which glycosylated chains with repeating units called glycosaminoglycans (GAG) are grafted. Depending on the type of skin (phanerogenic or glabrous) their distribution may be heterogeneous or homogeneous. They also occur as constituents of the plasmic membrane and some are directly associated with the collagen fibrils. Owing to their negative charges, they produce an environment round them capable of trapping ions, water and various metabolites. GAG chains also perform an essential function owing to their affinity for circulating growth factors.

Chondroitin sulphates (CS) are usually obtained from animal cartilage or skins and are characterised by the presence of a sulphate group on the disaccharide unit. Industrially, CS are obtained mainly from avian, ovine, bovine, porcine and shark cartilages.

In view of the risk of bovine spongiform encephalopathy, bovine and ovine cartilages are no longer used to a significant extent. It is also likely that in the near future a larger number of shark species will be classed as protected species.

### THE PRIOR ART

The methods of preparing crude CS, i.e. mixtures of different CS and use thereof in various forms for treatment of inflammation, scarring or other skin troubles are well-known, e.g. Patents EP 0063973 dated Nov. 3, 1982, U.S. Pat. No. 5,364,845 dated Nov. 15, 1994, FR 2756828 of Jun. 12, 1998, FR 2770777 of Nov. 10, 1997, WO 98/22114 of May 28, 1998, WO 98/26789 of Jun. 25, 1998, WO 98/27988 of Jul. 2, 1988 and U.S. Pat. No. 5,929,050 of Jul. 27, 1999.

U.S. Pat. No. 4,302,577 published on Nov. 24, 1981 relates to a method of preparing CS from bovine tracheas by dissolving them in concentrated soda, then by forming a chemical complex or conveying the solution over ion exchange resins. Various purification treatments are then applied. The total duration of the process is considerably above 72 hours, using expensive reagents. The uses of the products are therefore limited owing to the very high cost of obtaining them.

FR patent 2756828 published on Jun. 12, 1998 relates to a method of preparing CS from avian cartilage. The CS is obtained by enzymatic reaction using an alkalase for about 8 hours, followed by decantation and ultrafiltration. The filtrate is then subjected to hydrochloric hydrolysis for 5 to 6 hours in order to reduce the molecular weight of the CS. The solution is then neutralised with soda and the CS is isolated by precipitation and double washing with ethanol. The operation lasts about 24 hours.

All these processes use organic products or solvents during the purification phases, and are consequently lengthy and expensive.

In general, the CS compositions which have been developed are administered orally, except for treatment of open wounds (bruises or burns), in which the active principles can find access to the deep layers. On the one hand, oral administration takes account of the degradation of CS which occurs in organisms and is a means whereby molecules are conveyed to the target sites when their molecular weight has been reduced, and on the other hand local application of CS compositions in the case of open wounds or deep burns is justified by the degradation of CS macromolecules which occurs in situ. In the case however of local applications without surface damage to the skin, apart from the film-forming properties of CS, transcutaneous transport phenomena are ineffective in the case of macromolecules having a molecular weight comparable with that of CS, which covers a range from 25000 to 150000 daltons. It is therefore impossible, by direct external application of CS, to treat internal disorders of the skin such as subcutaneous inflammation, artificial or natural ageing, or other similar disturbances where the usual aim of treatment is to stimulate the metabolism of the fibroblasts and reconstitute the moisturising structures of the extracellular matrix, generally obtained via the availability in situ of active fragments of CS.

For all these reasons, it was therefore essential to develop a CS compound or CS fragments, preferably of low molecular weight, from raw materials coming from species which were neither protected nor in process of becoming protected, guaranteeing absence of contamination dangerous to man, in a process carried out exclusively in an aqueous medium by well-mastered industrial techniques which were inexpensive and of short duration and yielded the said CS or fragments of CS characterised by the precise metabolic activity for obtaining improved results in cosmetic skin applications.

### DESCRIPTION OF THE INVENTION

The invention relates to a low molecular weight chondroitin sulphate compound having cosmetic activity, characterised in particular by its efficient incorporation in vitro of thymadine, glucosamine and leucine in fibroblast molecules of the human cutis. Local application of this compound stimulates metabolism of mammalian fibroblasts including those of man. The invention also relates to a method of preparing the said compound.

In an embodiment of the invention, the raw materials come from marine animals, particularly cartilages of selachians such as spotted dog-fish, siki etc or bones of teleosts, such as fresh cod, coal-fish, burbot, halibut etc, particularly skate cartilages.

It is possible to use whole animals, but normally the raw materials used according to the invention are by-products obtained after commercial use of the edible parts of these species. These by-products consist of the cartilaginous parts proper and residues of skin mainly comprising protein constituents, fat and minerals. The method according to the invention therefore consists in crushing skate cartilages, introducing them into a reactor in the presence of demineralised water at a ratio of cartilage to water between 10% and 60% by weight, preferably between 20% and 50% by weight, and agitation at a temperature between 45° C. and 65° C., preferably between 55° C. and 60° C., at which temperature a protease (e.g. papain) is added, the reaction being continued for between 4 and 8 hours, preferably between 5 and 7 hours. The pH is then adjusted with concentrated sulphuric acid to a value between 0.7 and 2.2, preferably between 1.1 and 1.8, in order to precipitate the proteins and polypeptides and stop the enzyme reaction. Agitation is continued for between 20 and 40 minutes, then the assembly is neutralised with sodium hydroxide, obtaining a final pH between 6.5 and 8.5, preferably between 7.0 and 8.0. The mixture is then screened with a metal screen having a mesh size preferably around 200 microns. The dry matter in the suspension is adjusted with demineralised water to a concentration between 5% and 9% by weight, preferably between 6% and 8% by weight, to facilitate centrifugal decantation of the remaining insolubles in a horizontal centrifugal decanter (type SDA® by WESTFALIA). The next step is to eliminate the mineral burden together with all the low molecular weight compounds in the solution by diafiltration in demineralised water kept at 50° C. to avoid any microbiological contamination. This operation is advantageously effected with organic membranes in spirals (KOCH®) mounted in an ultrafiltration apparatus having a cut-off threshold between 2000 and 10000 daltons, preferably between 4000 and 8000 daltons. The progress of the operation is followed by measuring the conductivity of the water coming from the membranes. A value of 300 μSi is considered satisfactory. The residue after ultrafiltration is then filtered on cellulose plates containing active carbon mounted in a plate filter so as to complete the discoloration and deodorisation of the residue. The filtrate is then concentrated by evaporation of water in vacuo at temperature of about 50° C., the evaporator being equipped with a venturi for injecting a neutral gas such as nitrogen, helium or argon, for optimum deodorisation. The neutral gas entrains the odoriferous volatile components. The concentrate is dried in a freeze-drying apparatus. The yield of dry/crude matter is about 2.5%. The substance obtained has a CS purity greater than 95%. These CS, in the form of the sodium salt, are characterised by molecular weights above 100000 daltons.

In another embodiment of the invention, the residue after ultrafiltration (diafiltration) is brought to a content of dry matter near 1%, using demineralised water, then transferred to a vitrified reactor in which the CS solution is acidified under vigorous agitation with sulphuric acid until the solution is brought to a normality between 1 and 2, preferably between 1.4 and 1.6. The temperature of the mixture is then progressively brought by injection of live steam to a temperature between 50° C. and 70° C., preferably between 55° C. and 65° C. This step is for the purpose of careful hydrolysis of the CS, a process which determines the future properties of the low molecular weight CS. This step is very tricky, since it is essential to avoid rearrangements or reorientation of the chains which often occurs in the case of pseudoplastic solutions. This depolymerisation is followed in an Ubbelohde type viscosimeter by measuring the flow times of the solution. The reaction is stopped when the starting solution, in a viscometer equipped with a capillary having an internal diameter of 0.5 mm, has a flow time between 90 and 110 seconds, preferably between 95 and 105 seconds. These flow times are obtained in a reaction with a total duration between 100 minutes and 140 minutes, preferably between 110 and 130 minutes.

The solution is neutralised with calcium hydroxide. The suspension is then cooled, yielding a precipitate in the form of calcium sulphate which can easily be eliminated by centrifugal decantation. The supernatant is then diafiltered on spiral membranes having a cut-off threshold of 1000 daltons (OSMONICS) at elevated pressure, in a supply of demineralised water at a temperature of 50° C. As before, the progress of the operation is followed by measuring the resistivity of the permeate; values below 100 μSi indicate that the demineralisation of the solution of low molecular weight chondroitin sulphate solution “CSBP” is effective. The amount of diafiltration water required in this operation is about five times the volume of the initial solution for diafiltering. The yield of freeze-dried CSBP is about 50% of the quantity of dry matter used in the hydrolysis reaction.

In a preferred embodiment of the invention, a better yield of CSBP can be obtained, with better control of the depolymerisation reaction avoiding the problems of rearrangement of chains, a much lower quantity of diafiltration water and a decrease in the required surface area of the diafiltration membranes or in the duration of this step, by preliminary acidification of the CS solution by passing it over ion exchange resins. Advantageously next, a CS solution containing between about 2% and 6%, preferably between 3% and 5% dry matter is passed over a column containing a strong cationic resin (e.g. IR 220 by ROHM and HAAS) under conditions for obtaining a CS solution having a pH near 1. This advantageously avoids the step of adding sulphuric acid in the vitrified reactor, which is tricky owing to the precise viscosimetric flow speed characteristics required at the end of the reaction. Under these conditions, furthermore, the partial depolymerisation reaction is much slower, so that the characteristics of the CSBP are easier to control. After heating the CS solution to a temperature between 60° C. and 80° C., preferably between 65° C. and 75° C. by injection of live steam, the CS hydrolysis reaction is continued for between 10 hours and 14 hours, preferably between 9 hours and 12 hours, so as to obtain a flow speed in the capillary viscosimeter 2.5 to 3.0 times lower than that obtained with the native CS according to the invention. The solution is then cooled to a temperature between 30° C. and 40° C., then brought to a pH near 6.0 with sodium hydroxide and then subjected to the diafiltration step on diaphragms having a cut-off threshold of 1000 daltons. In another embodiment of the invention, neutralisation is effected with hydroxides of zinc, manganese, lysine or magnesium. The various steps of this preferred embodiment advantageously enable only two volumes of water per initial volume of CSBP to be used in this step, the yield of CSBP dry matter after freeze-drying being near 80%.

This embodiment of the invention provides a CSBP substance having an average molecular weight, determined by high-pressure size exclusion chromatography HPSEC (for example on a TSK SW 3000XL column from TOSOHAS) lower than 15000 daltons, by comparison with pullulans, native chondroitin sulphate and sugar standards having various molar weights.

The following non-limitative example is described so as to illustrate the invention.

### EXAMPLE

In an enzymatic reaction, 400 kg of crushed skate cartilages was introduced into 600 litres of demineralised water. The temperature was brought to 58° C. by injection of steam. 2 kg of papain was then added and the reaction was allowed to proceed for 6 hours with agitation. The pH was then adjusted to 1.2 with 96% sulphuric acid. After agitation for 30 minutes, the mixture was neutralised with 30.5% soda, giving a pH of 7.5. The mixture was screened on a screen equipped with cloth having a mesh size of 200 microns, then centrifuged in an SDA 230 centrifugal decanter. The supernatant was then diafiltered in an ultrafiltration device equipped with KOCH spiral membranes having a cut-off power between 30000 and 50000. After 2840 litres of demineralised water had passed through, 472 kg of liquid solution containing 2.25% dry matter was recovered and concentrated in an evaporator, yielding 465 kg containing 4% dry matter. An aliquot part of the solution was freeze-dried, showing that the resulting native chondroitin sulphate had a purity higher than 95%. A 2 mg/ml solution of this product and solutions of similar concentration of two commercial products were passed through an HPSEC apparatus equipped with a TSK SW 3000 XL (TOSOHAS) column with a 0.1 M ammonium acetate eluent, and yielded the following results (average molecular weights) after calibration with pullulans having various molecular weights:

| | |
|---|---|
| Commercial native CS No. 1: | 40000 daltons |
| Commercial native CS No. 2: | 50000 daltons |
| Native CS in the example: | 130000 daltons |

250 litres of the 4% solution were conveyed through a column containing 7 litres of IR120 resin (ROHM and HAAS). 250 litres at pH 1.0 and with 3.8% dry matter were obtained at the outlet. For the purpose of partial depolymerisation of the resulting CS, 250 litres of solution with 250 litres of demineralised water were placed in a vitrified reactor for 10 hours, with agitation, after raising the temperature of the mixture to 70° C. At the end of the depolymerisation reaction, 3.75 litres of 30.5% sodium hydroxide were added, bringing the pH to 6.0. The assembly was then introduced into the diafiltration apparatus equipped with spiral membranes (KOCH®) having a cut-off threshold of 1000 daltons. After 750 litres of demineralised water at 50° C. had flowed through, 400 litres of residue were obtained with 120 μSi conductivity. After filtration on cellulose plates with active carbon, followed by concentration and deodorisation by injection of nitrogen, 80 litres of solution containing 10% dry matter were obtained, a 2% yield from the starting raw material. The following result was obtained for the average molecular weight by exclusion chromatography of the freeze-dried product under the same conditions as before:

CSBP according to the invention: 10000 daltons

The previously-described capillary flow tests showed that the product had a flow speed 2.8 times slower than the native CS according to the invention and 1.9 times slower than a commercial native CS. The resulting depolymerised CS had a purity above 95%.

In another embodiment of the invention the CSBP obtained in the present example was characterised by its efficiency in stimulating the fundamental metabolism of human dermal fibroblasts, more particularly by incorporation of various markers such as thymidine (cell growth), leucine (protein synthesis) and glucosamine (synthesis of GAG).

Tests in vitro on human fibroblast cultures are a means of evaluating the capacity of CSBP to stimulate the fundamental metabolism of the extracellular matrix of the cutis. Under conventional conditions of cultivation in triplicate, the CSBP according to the invention and positive references (EGF, epidermis growth factor at 10 ng/ml, TGF-β human transformation factor b at 10 ng/ml and vitamin C at 20 mg/ml) were placed in contact with fibroblasts. The neosynthesis can be quantified by radioactive labelling of the specific metabolites (amino acids). Two concentrations of CSBP were tested: 2 and 0.4 mg/ml. The results obtained in the following table were subjected to the Dunnett test:

| MARKERS | REFERENCE | CSBP 0.4 mg/ml | CSBP 2.0 mg/ml |
|---|---|---|---|
| % Thymidine | EGF: 100 | 74 p < 0.01 | 183 p < 0.01 |
| | p < 0.01 | | |
| % Leucine | VIT C: 45 | 44 p < 0.05 | 88 p < 0.01 |
| | p < 0.05 | | |
| % Glucosamine | TGF: 200 | 57 p < 0.01 | 178 p < 0.01 |
| | p < 0.01 | | |

The culture conditions were as follows:

Culture medium MEM/M199: sodium bicarbonate 1.87 mg/ml, L-glutamine 2 mM, penicillin 50 IU/ml and foetal calf serum 10%.

Temperature: 37° C.

Atmosphere: 5% CO₂

These tests show a characteristic effect of the CSBP according to the invention on the growth of human fibroblasts under the various metabolic conditions which govern the physiological equilibrium of the cutis and indirectly contribute to the equilibrium of the epidermis.

Formulations for application comprise inter alia gels and emulsions of CSBP in the form of salts of sodium, zinc, manganese or magnesium.

1. A low molecular weight chondroitin sulphate compound (CSBP) extracted from cartilages of marine animals by a process conducted exclusively in the aqueous phase, consisting mainly in enzymatic hydrolysis, acid depolymerisation and diafiltration, characterised in that the said CSBP can be used in vitro, under defined culture conditions and at a concentration of 0.2%, to obtain at least one of the following results:
(i) increasing the cell growth of human dermal fibroblasts by more than 170%, determined by incorporation of thymidine;
(ii) increasing the cell growth of human dermal fibroblasts by more than 160%, determined by incorporation of glucosamine, and
(iii) increasing the cell growth of human dermal fibroblasts by more than 70%, determined by incorporation of leucine.

2. A CSBP compound according to claim 1, characterised in that the compound is in the form of the salt of sodium, zinc, magnesium or manganese.

3. A CSBP compound according to claims 1 and 2, characterised in that it is obtained from the cartilages of skate.

4. A CSBP compound according to claims 1 to 3, characterised in that its molecular weight is less than 15,000 daltons.

5. A method of preparing the CSBP compound according to claims 1 to 3, characterised in that the crushed cartilages are introduced into a reactor at a ratio of cartilage to water between 10% and 60% by weight, preferably between 20% and 50% by weight.

6. A method of preparing the CSBP compound according to claim 5, characterised in that the enzyme reaction is effected with a papain protease.

7. A method according to claim 6, characterised in that the enzyme reaction is performed at a temperature between 45° C. and 65° C., preferably between 55° C. and 60° C.

8. A method according to claim 7, characterised in that the enzymatic reaction lasts between 4 and 8 hours, preferably between 5 and 7 hours.

9. A method according to claim 8, characterised in that the pH is adjusted to a value between 0.7 and 2.2, preferably between 1.1 and 1.8.

10. A method according to claim 9, characterised in that the pH is then adjusted to a value between 6.5 and 8.5, preferably between 7.0 and 8.0.

11. A method according to claim 10, characterised in that the solution is decanted.

12. A method according to claim 11, characterised in that the decanted solution is diafiltered on organic membranes having a cut-off threshold between 2000 and 10000 daltons, preferably between 4000 an 8000 daltons.

13. CS obtained by working the method according to any of claims 5 to 12, characterised in that its molecular weight is above 100000 daltons.

14. A method according to claim 12, characterised in that the dry matter in the aqueous solution is between 2% and 6%, preferably between 3% and 5%.

15. A method according to claim 14, characterised in that the solution is acidified by passing over an ion exchange resin.

16. A method according to claim 15, in that the resin is a strong cationic resin.

17. A method according to claim 16, characterised in that the solution coming from the resin is brought to a temperature between 60° C. and 80° C., preferably between 65° C. and 75° C.

18. A method according to claim 17, characterised in that the temperature is maintained for between 10 hours and 14 hours, preferably between 9 hours and 12 hours.

19. A method according to claim 18, characterised in that the pH of the solution is brought to a value of 6.0 after cooling to a temperature between 30° C. and 40° C.

20. A method according to claim 19, characterised in that the neutralisation agent is a hydroxide of sodium, zinc, manganese, lysine or magnesium.

21. A method according to claim 20, characterised in that the solution is diafiltered on organic membranes having a cut-off threshold of 1000 daltons at most.

22. A method according to claim 21, characterised in that the diafiltered solution is deodorised in vacuo by injecting a neutral gas.

23. Use of a low molecular weight chondroitin sulphate compound according to claims 1 to 4 in cosmetic preparations for use on the skin.
